(19) United States
(12) Patent Application Publication
(10) Pub. No.: US 2026/0207903 A1
(43) Pub. Date: Jul. 23, 2026

(54) MEDICAL DEVICE

(71) Applicant: ASAHI INTECC CO., LTD., Seto-shi (JP)

(72) Inventors: Hiroaki TAKAHASHI, Aichi (JP); Yukihiro FUSEYA, Aichi (JP)

(73) Assignee: ASAHI INTECC CO., LTD., Seto-shi (JP)

(21) Appl. No.: 19/570,234

(22) Filed: Mar. 18, 2026

Related U.S. Application Data
(63) Continuation of application No. PCT/JP2024/029357, filed on Aug. 19, 2024.

(30) Foreign Application Priority Data
Sep. 22, 2023 (JP) 2023-158123

(51) Int. Cl. A61M 29/00

(52) U.S. Cl. CPC A61M 29/00; A61M 2207/00

(57) ABSTRACT

A medical device capable of sending and suctioning a liquid includes a hollow shaft with a coil. The coil is a wire wound around a long axis. The hollow shaft has an inner space on a radially inward side of the coil along a long axis direction. A spiral protrusion is provided on an outer peripheral surface of the hollow shaft. A fluid-delivery structure is attached to the hollow shaft and forms a liquid-tight lumen within the inner space

### CROSS-REFERENCE TO RELATED APPLICATIONS

This application is a continuation application of International Application No. PCT/JP2024/029357, filed Aug. 19, 2024, which claims priority to Japanese Patent Application No. 2023-158123, filed Sep. 22, 2023. The contents of these applications are incorporated herein by reference in their entirety.

### TECHNICAL FIELD

The present disclosure relates to a medical device.

### BACKGROUND

As the medical device, for example, a dilator used to dilate a hole formed in a body, a catheter used to treat a lesion in a body cavity, and the like are known.

In such a medical device, a technique of forming a hollow shaft with a coil is known (for example, see Japanese Unexamined Patent Publication No. 2007-98120).

### SUMMARY

A medical device includes: a hollow shaft that includes a coil formed of wire wound around a long axis, a spiral protrusion, and a fluid-delivery structure. The hollow shaft has an inner space on a radially inward side of the coil along a long axis direction. The spiral protrusion is provided on an outer peripheral surface of the hollow shaft. The fluid-delivery structure is attached to the hollow shaft and is configured to form a liquid-tight lumen within the inner space.

As used herein, the “long axis” refers to a center axis along the long axis direction of a hollow shaft (e.g., an axis z in FIGS. 1 and 5), unless otherwise specified. The “distal end side” refers to the direction along the long axis (long axis direction) and the direction (distal side) in which the medical device is advanced toward a treatment site. The “proximal end side” refers to the direction along the long axis (long axis direction) and the direction (proximal side) opposite to the above-described distal end side. The “distal end” refers to an end portion on the distal end side of an arbitrary member or portion, and the “proximal end” refers to an end portion on the proximal end side of an arbitrary member or portion. The “radial direction” refers to a direction orthogonal to the long axis.

### BRIEF DESCRIPTION OF THE DRAWINGS

FIG. 1 is a schematic side view illustrating a first embodiment.

FIG. 2A is a schematic side view of an exploded medical device in FIG. 1, illustrating an integrated structure including a distal tip, a hollow shaft, and a spirally-arranged protruding portion.

FIG. 2B is a schematic side view of the exploded medical device in FIG. 1, illustrating an integrated structure including a tube and a grip portion.

FIG. 3 is a schematic sectional view illustrating an enlarged distal end portion in FIG. 1.

FIG. 4A is a schematic sectional view illustrating an enlarged distal end portion according to a modification of the first embodiment.

FIG. 4B is a schematic sectional view illustrating an enlarged distal end portion according to a modification of the first embodiment.

FIG. 4C is a schematic sectional view illustrating an enlarged distal end portion according to a modification of the first embodiment.

FIG. 5 is a schematic side view illustrating a second embodiment.

FIG. 6 is a schematic sectional view illustrating an enlarged distal end portion in FIG. 5.

FIG. 7A is a schematic sectional view illustrating an enlarged distal end portion according to a modification of the second embodiment.

FIG. 7B is a schematic sectional view illustrating an enlarged distal end portion according to a modification of the second embodiment.

FIG. 7C is a schematic sectional view illustrating an enlarged distal end portion according to a modification of the second embodiment.

### DETAILED DESCRIPTION

Hereinafter, embodiments of the present disclosure will be described with reference to the drawings. The present disclosure is not limited to the embodiments described in the drawings. The dimensions of each part illustrated in the drawings are the dimensions illustrated to facilitate understanding of the embodiments, and do not necessarily correspond to the actual dimensions.

In each drawing, the left side in the drawing is a distal end side (distal side) that is inserted into a deeper portion in a body, and the right side is a proximal end side (proximal side, hand side) that is operated by a professional such as a physician.

When performing a procedure using the above-described medical device, a physician sends a liquid such as a contrast medium or a drug solution into a body cavity, or suctions an excessive body fluid such as bile accumulated in a body. At this time, in the hollow shaft formed of the coil, since the liquid can enter and exit through a space of the coil, the physician needs to temporarily pull out the medical device formed of the coil to the outside of the body, and then insert a liquid-tight catheter to a site to be treated to perform liquid sending or suction.

The present disclosure has been made in view of the above-described circumstances and provides a medical device capable of sending and suctioning a liquid while forming a hollow shaft with a coil. [0011]

### First Embodiment

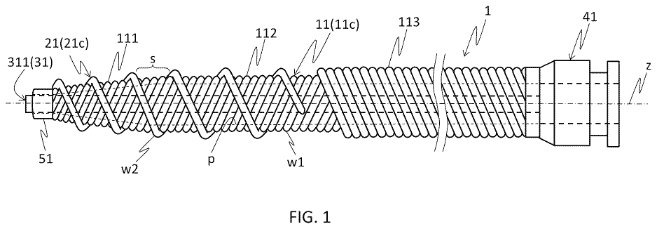

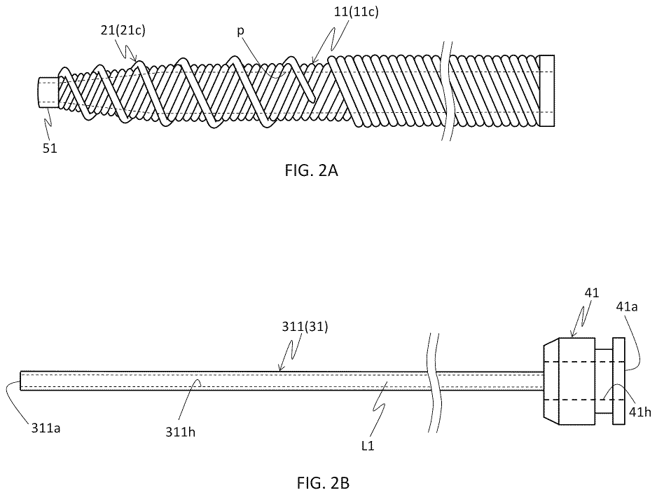

FIGS. 1 to 3 are schematic views illustrating a first embodiment. The medical device 1 is a dilator. As illustrated in FIGS. 1, 2A, and 2B, the medical device 1 generally includes a hollow shaft 11, a spirally-arranged protruding portion 21, a lumen forming body 31, a grip member 41, and a distal tip 51.

The hollow shaft 11 is a member that is formed of a coil in which a wire is wound around the long axis and has an inner space on the radially inward side of the coil along the long axis direction.

The hollow shaft includes a tapered portion 111, a large diameter portion 112, and a main body portion 113. The tapered portion 111 is a tapered portion having a shape tapered toward the distal end side. The large diameter portion 112 is a portion that has a distal end located at the proximal end of the tapered portion 111, extends toward the proximal end side in the long axis direction, and has a substantially constant outer diameter. The main body portion 113 is a portion that has a distal end located at the proximal end of the large diameter portion 112 and that extends toward the proximal end side in the long axis direction.

Each of the tapered portion 111, the large diameter portion 112, and the main body portion 113 is formed by appropriately adjusting the shape of the outermost periphery of the coil (hereinafter, also referred to as a “first coil 11c”) (the diameter of the first coil 11c) forming the hollow shaft 11 along the long axis direction. The first coil 11c is formed by spirally winding a wire w1 so that the wires w1 adjacent to each other along the long axis direction of the hollow shaft 11 are in close contact with each other.

The first coil 11c may be either a single-thread coil formed by winding a single wire or a multi-thread coil formed by winding two or more wires. Examples of the wire include a single wire (one single wire) and a twisted wire (a bundle of wire groups formed by twisting two or more single wires together in advance).

Since the first coil 11c is inserted into a body cavity, the first coil 11c may be antithrombogenic, flexible, and biocompatible. Examples of the material of the wire w1 forming the first coil 11c include resin materials such as polyamide resins, polyolefin resins, polyester resins, polyurethane resins, silicone resins, and fluororesins, and metallic materials such as stainless steels (e.g., SUS304) and superelastic alloys (e.g., nickel-titanium alloys).

As described above, since the hollow shaft 11 is formed of the first coil w1 in which the wire 11c is wound around the long axis, it is possible to improve the flexibility of the hollow shaft 11 and the torquability of the first coil 11c. The torquability is the ability to transmit a rotational force applied to the proximal end portion (the grip member 41 described below according to the present embodiment) of the hollow shaft 11 to the distal end portion of the hollow shaft 11.

The spirally-arranged protruding portion 21 (also referred to as “spiral protrusion”) is a member that is provided on the outer peripheral surface of the hollow shaft and has a space between adjacent portions along the long axis direction of the hollow shaft. The spirally-arranged protruding portion 21 can be formed of a coil (hereinafter, also referred to as a “second coil 21c”). For example, the second coil 21c may be provided to protrude toward the outer side in the radial direction of the hollow shaft 11 from the outer peripheral surface of the hollow shaft 11 by winding a wire w2 around the outer peripheral surface of the hollow shaft 11 (the outer peripheral surfaces of the tapered portion 111 and the large diameter portion 112 according to the present embodiment) and to have a space s by separating the adjacent wires w2 from each other in the long axis direction. In some embodiments, the spiral protrusion 21 is a separate wire wound over the outer peripheral surface of the hollow shaft 11 and attached thereto. This attachment may be achieved via mechanical tension, adhesive bonding, or thermal joining such as welding or brazing at the distal and proximal ends. In alternative embodiments, the spiral protrusion 21 may be integrally formed with the hollow shaft 11.

Since the second coil 21c is inserted into a body cavity, the second coil 21c may be antithrombogenic, flexible, and biocompatible. Examples of the material of the wire w2 forming the second coil 21c include the same materials as those exemplified as the material of the wire w1 forming the first coil 11c.

The first coil 11c and the second coil 21c can be joined to each other by, for example, brazing, welding, fixing with an adhesive, welding with coating, or the like, at the end portions thereof.

As described above, since the spirally-arranged protruding portion 21 is provided on the outer peripheral surface of the hollow shaft 11 and has the space s between the adjacent portions along the long axis direction of the hollow shaft 11, the medical device 1 can be advanced by the rotation operation due to the screw action of the spirally-arranged protruding portion 21. The spirally-arranged protruding portion 21 does not hinder the forward movement by a pushing operation as long as it is not engaged with the inner peripheral surface of the body cavity.

The lumen forming body 31 (also referred to herein as a fluid-delivery structure) is attached to the hollow shaft and forms a liquid-tight lumen on the radially inward side of the hollow shaft. As used herein, “liquid-tight” refers to a seal or boundary that prevents the escape of medical fluids, such as contrast media or drug solutions, at standard operating pressures encountered during vascular or endoscopic procedures The lumen forming body 31 according to the present embodiment is a liquid-tight tube 311 that is provided in an inner space p of the first coil 11c and extends along the long axis direction. The tube 311 includes an inner cavity 311h having an opening portion 311a at the distal end. The inner cavity 311h is kept to be liquid-tight so that the liquid does not leak to the outside of the tube 311 or does not flow in from the outside. Thus, the medical device 1 has high liquid tightness with a simple configuration.

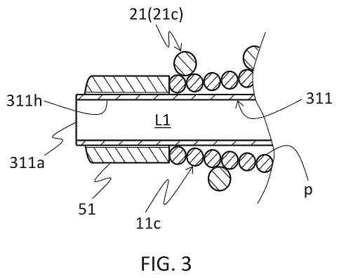

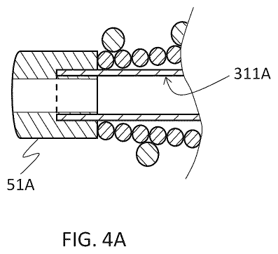

The outer peripheral surface of the tube 311 may be in contact with or may be separated from, e.g., have a clearance between, the inner peripheral surface of the first coil 11c. As illustrated in FIG. 3, according to the present embodiment, the outer peripheral surface of the tube 311 abuts on the inner peripheral surface of the distal end portion of the first coil 11c, and the distal end portion of the tube 311 is provided to protrude from the distal end of the distal tip 51 through the distal tip 51 described below.

The tube 311 and the hollow shaft 11 may be coupled so as not to be detachable. The tube 311 and the hollow shaft 11 may be coupled to each other via the grip member 41 by, for example, fixing each of the tube 311 and the first coil 11c to the grip member 41. Thus, the tube 311 and the hollow shaft 11 can always be used integrally.

The tube 311 and the hollow shaft 11 may be coupled so as to be detachable. The mechanism for attachment and detachment is not particularly limited. For example, the grip member to which the tube is fixed may be coupled to the first coil 11c by using an attaching and detaching mechanism. As a result, the tube 311 can be inserted into the hollow shaft 11 or both can be separated from each other as needed, and liquid sending of a contrast medium or the like and suction of a body fluid or the like can be performed as appropriate.

Since the tube 311 is inserted into a body cavity, the material forming the tube 311 may be antithrombogenic, flexible, and biocompatible. The material forming the tube 311 may be flexible to be able to follow a curved body cavity. Examples of the material forming the tube 311 include polyamide resins, polyolefin resins, polyester resins, polyurethane resins, silicone resins, fluororesins, polyacrylic acids, polyimide resins, and polyamide-imide resins.

The grip member 41 is a member used for the medical device 1 to be pushed into the body or rotated by the professional. The grip member 41 according to the present embodiment has an inner cavity 41h having an opening portion 41a at the proximal end and is fixed to the proximal end portion of the tube 311. The distal end of the lumen-forming body 31 is fluidly coupled to the opening 41h of the distal tip 41, creating a continuous, sealed fluid pathway from the proximal end to the distal-most exit of the device.

The inner cavity 311h of the tube 311 and the inner cavity 41h of the grip member 41 communicate with each other, and a lumen L1 is formed by the inner cavity 311h and the inner cavity 41h. For example, a guide wire is inserted into the lumen L1 during a procedure, or a contrast medium to be injected into a body cavity or a liquid such as a body fluid suctioned from a body cavity flows through the lumen L1.

The proximal end of the distal tip 51 is fixed to the distal end of the hollow shaft 11. The distal tip 51 is a substantially hollow cylindrical member extending from the distal end of the hollow shaft 11 toward the distal end side. The distal tip 51 may have, for example, a flatter surface than the outer peripheral surface of the first coil 11c (the uneven surface formed by the wire w1) and may be formed to have a hollow shape so that the tube 311 can be inserted. The distal tip 51 can be formed, for example, at the distal end portion of the first coil 11c by forming its surface into a flat shape using a brazing material such as silver-tin brazing or gold-tin brazing. As a result, the insertability of the hollow shaft 11 into a hole drilled in advance can be improved, and the procedure can proceed smoothly.

The distal end portion of the hollow shaft 11 may be provided to be rotatable around the long axis with respect to the tube 311. The inner peripheral surface of the distal end portion of the first coil 11c and the outer peripheral surface of the tube 311 do not need to be fixed (joined) to each other, and the first coil 11c may be provided to be freely rotatable around the tube 311. As a result, high torquability can be maintained up to the distal end portion of the medical device 1. According to the present embodiment, the inner peripheral surface of the distal tip 51 is not fixed to the outer peripheral surface of the tube 311. As a result, the first coil 11c and the distal tip 51 can freely rotate integrally around the tube 311.

An example of the use mode of the medical device 1 exemplified as a medical device will be described.

First, the physician uses an introducer needle to penetrate and puncture the target body tissue. Subsequently, the physician inserts a guide wire into the inner cavity of the introducer needle and then removes the introducer needle from the body.

Subsequently, the physician inserts the proximal end of the guide wire into the inner cavity 311h from the opening portion 311a of the tube 311 and then advances the medical device 1 in the body cavity along the guide wire to insert the distal tip 51 into the hole. Subsequently, the physician expands the hole by advancing the medical device 1 while rotating the hollow shaft 11 by operating the grip member 41. At this time, since the tapered portion 111 advances to the distal end side while passing through the hole by the screw action or the like of the spirally-arranged protruding portion 21 (the second coil 21c) due to the rotation of the hollow shaft 11, the hole is expanded by the tapered portion 111.

Subsequently, the physician sends a liquid such as a contrast medium or suctions a liquid such as a body fluid through the lumen L1. To be specific, the physician couples a syringe (e.g., a contrast medium injection device or a liquid suction device) to the opening portion 41a of the grip member 41 using a coupling member such as a Luer lock, and sends or suctions a liquid through the lumen L1. At this time, since the tube 311 is provided such that the inner cavity 311h forms the liquid-tight lumen L1, the liquid in the lumen L1 does not leak to the outside, and liquid sending and suction are performed.

After the liquid sending and the suction are completed, the physician retracts the medical device 1 while reversely rotating the hollow shaft 11 by the operation of the grip member 41 and removes the medical device 1 to the outside of the body.

As described above, since the medical device 1 has the above-described configuration, the medical device 1 can exhibit excellent torquability and has a function of sending a liquid such as a contrast medium or suctioning a liquid such as a body fluid through the lumen L1 formed in a liquid-tight manner by the tube 311.

Unlike the above-described embodiment, the tube 311 and the hollow shaft 11 may be detachably coupled to each other using, for example, a known attaching and detaching mechanism so that the tube and the hollow shaft can be attached to and detached from each other.

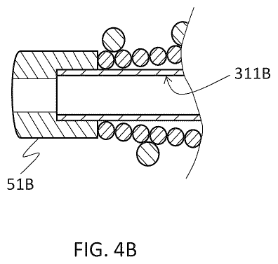

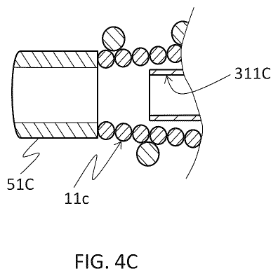

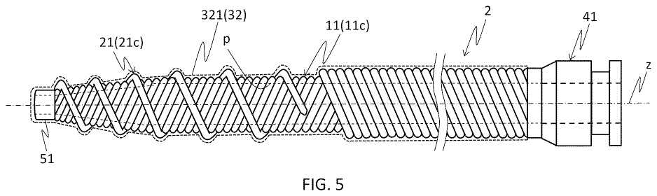

Unlike the above-described embodiment, the distal end portion of the tube may be provided so as not to protrude from the distal tip. For example, as illustrated in FIGS. 4A and 4B, distal end portions of tubes 311A and 311B may be provided to simply abut on distal tips 51A and 51B without being fixed thereto. As illustrated in FIG. 4C, the distal end portion of a tube 311C may be provided away from a proximal end of a distal tip 51C toward the proximal end side. That is, the distal end of the tube 311C may be located at an intermediate position of the first coil 11c in the long axis direction.

### Second Embodiment

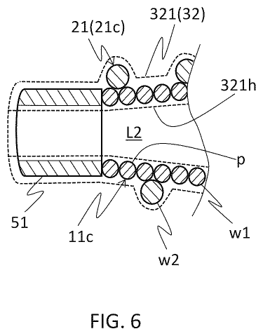

FIGS. 5 and 6 are schematic views illustrating a second embodiment. The medical device 2 is a dilator. As illustrated in FIG. 5, the medical device 2 schematically includes the hollow shaft 11, the spirally-arranged protruding portion 21, a lumen forming body 32, the grip member 41, and the distal tip 51. The medical device 2 according to the present embodiment is different from the first embodiment in the configuration of the lumen forming body 32. Since the configurations of the hollow shaft 11, the spirally-arranged protruding portion 21, the grip member 41, and the distal tip 51 are the same as those in the first embodiment, the same portions are denoted by the same symbols, and the detailed description thereof will be omitted. Since the usage mode of the medical device 2 is the same as that of the first embodiment, a detailed description thereof will also be omitted.

The lumen forming body 32 is attached to the hollow shaft 11 and is provided to form a liquid-tight lumen. The lumen forming body 32 according to the present embodiment is a coating 321 applied to the hollow shaft 11.

The coating 321 is formed to cover at least a part of the hollow shaft 11 so that a liquid does not flow in or out at least through the interval between the adjacent wires w1 of the first coil 11c. According to the present embodiment, an inner cavity 321h formed on the radially inward side of the coating 321 forms a liquid-tight lumen L2. For example, as illustrated in FIG. 6, the portion where the coating 321 is provided is a portion that covers the entirety of the first coil 11c, the spirally-arranged protruding portion 21, and the distal tip 51. The material of the coating 321 infiltrates and fills the spaces between adjacent turns of the wire w1 of the first coil 11c, thereby sealing the gaps between the turns. This sealing ensures that the lumen L2 remains liquid-tight even when a pressurized fluid, such as a contrast medium, is introduced into the inner space p.

From the viewpoint of ensuring the torquability, the material of the coating 321, the thickness of the coating 321, and the like may be determined such that the relative movement between the adjacent wires w1 of the first coil 11c is not hindered. Since the coating 321 is inserted into a body cavity, the material may be antithrombogenic, flexible, and biocompatible.

Examples of the material forming the coating 321 include resins such as polyamide resins, polyolefin resins, polyester resins, polyurethane resins, silicone resins, fluororesins, polyacrylic acid, polyimide resins, and polyamide-imide resins.

As described above, since the medical device 2 has the above-described configuration, the medical device 2 can be operated while exhibiting excellent torquability, and provides a function of sending a liquid such as a contrast medium or suctioning a liquid such as a body fluid through the lumen L2 formed in a liquid-tight manner with the coating 321.

In the medical device 2, since the lumen forming body 32 is formed of the coating 321, the wide inner cavity 321h can be secured in the inner space p by reducing the thickness of the coating 321 while maintaining the liquid tightness. As a result, the medical device 2 has a function of smoothly sending and suctioning the liquid.

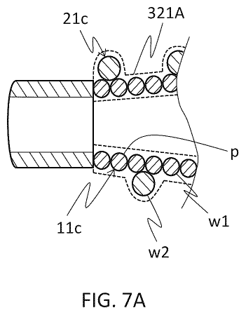

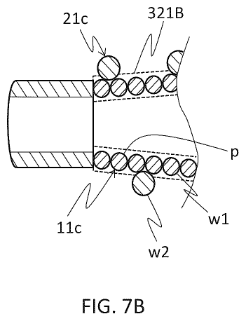

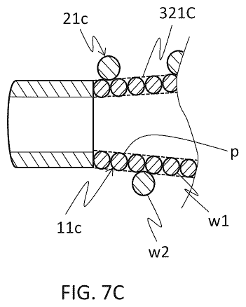

The portion where the coating is provided is not limited to the portion according to the above-described embodiment. Examples of the portion where the coating (321A, 321B, 321C) is provided include a portion that covers only the entirety of the first coil 11c and the second coil 21c (see FIG. 7A), a portion that covers only the entirety of the first coil 11c (see FIG. 7B), and a portion that covers the interval between the adjacent wires w1 of the first coil 11c (see FIG. 7C).

The present disclosure is not limited to the configurations according to the above-described embodiments, but is defined by the scope of the claims, and is intended to include all modifications within the meaning and scope equivalent to the scope of the claims. A part of the configuration according to the above-described embodiment may be deleted or replaced with another configuration, and another configuration may be added to the configuration according to the above-described embodiment.

For example, unlike the embodiments described above, the medical device may be a device (e.g., catheter) which have a substantially constant outer diameter along the long axis.

Unlike the embodiment described above, the medical device may be a device which includes both a tube and a coating. Examples of such a medical device include a medical device in which the distal end portion of the tube is provided to be separated from the proximal end of the distal tip toward the proximal end side and at least a portion of the first coil located between the proximal end of the distal tip and the distal end of the tube is covered with a coating. That is, the distal end of the tube may be located at an intermediate position of the first coil in the long axis direction.

Some aspects of the present disclosure provide the followings.
(1) A medical device including: a hollow shaft that is formed of a coil in which a wire is wound around a long axis and has an inner space on a radially inward side of the coil along a long axis direction; a spirally-arranged protruding portion provided on an outer peripheral surface of the hollow shaft; and a lumen forming body that is attached to the hollow shaft and is provided to form a liquid-tight lumen.
(2) The medical device according to (1), wherein the lumen forming body is a tube that is provided in the inner space and extends along the long axis direction.
(3) The medical device according to (2), wherein a distal end portion of the hollow shaft is rotatable around the long axis with respect to the tube.
(4) The medical device according to (2) or (3), wherein the tube and the hollow shaft are coupled so as not to be detachable.
(5) The medical device according to (2) or (3), wherein the tube and the hollow shaft are coupled so as to be detachable.
(6) The medical device according to (1), wherein the lumen forming body is a coating applied to the hollow shaft.

1. A medical device comprising:
a hollow shaft including a coil formed of a wire wound around a long axis, the hollow shaft having an inner space on a radially inward side of the coil along a long axis direction;
a spiral protrusion provided on an outer peripheral surface of the hollow shaft; and
a fluid-delivery structure attached to the hollow shaft and configured to form a liquid-tight lumen within the inner space.

2. The medical device according to claim 1, wherein the fluid-delivery structure includes a tube disposed in the inner space and extending along the long axis direction.

3. The medical device according to claim 2, wherein a distal end portion of the hollow shaft is rotatable around the long axis relative to the tube.

4. The medical device according to claim 2, wherein the tube and the hollow shaft are non-detachably coupled.

5. The medical device according to claim 2, wherein the tube and the hollow shaft are detachably coupled.

6. The medical device according to claim 2, wherein the tube includes a resin material and extends distally beyond a distal end of the hollow shaft.

7. The medical device according to claim 1, wherein the fluid-delivery structure includes a coating applied to the hollow shaft.

8. The medical device according to claim 1, further comprising a distal tip fixed to a distal end of the hollow shaft, the distal tip having a smoother outer surface than the outer peripheral surface of the hollow shaft.

9. The medical device according to claim 8, wherein the distal tip includes a brazing material that forms a substantially flat outer surface transitioning from the hollow shaft.

10. The medical device according to claim 1, wherein the wire of the coil includes a twisted wire formed of a plurality of wire groups.

11. The medical device according to claim 1, wherein the fluid-delivery structure includes a coating that seals gaps between adjacent turns of the wire of the coil.

12. A medical dilator comprising:
a coiled shaft having a distal tapered portion and an inner cavity;
a second coil wound around an exterior of the coiled shaft to form a screw-thread; and
a liquid-tight tube disposed within the inner cavity, the liquid-tight tube being rotatable relative to the coiled shaft such that the coiled shaft is configured to rotate around the liquid-tight tube.

13. The medical dilator of claim 12, wherein the liquid-tight tube is fixed at a proximal end of the medical dilator and is free at a distal end of the medical dilator.

14. The medical dilator of claim 12, wherein a clearance exists between an outer peripheral surface of the liquid-tight tube and an inner peripheral surface of the coiled shaft.

15. The medical dilator of claim 12, wherein the second coil has a larger wire diameter than a wire diameter of the coiled shaft.

16. The medical dilator of claim 12, wherein the liquid-tight tube is configured to transport a contrast medium.

17. The medical dilator of claim 12, wherein the distal tapered portion of the coiled shaft is formed by reducing a winding diameter of the coiled shaft toward a distal direction.

18. A method of manufacturing a medical device, the method comprising:
winding a first wire to form a hollow coiled shaft having an internal space;
winding a second wire around an outer surface of the hollow coiled shaft to form a spiral protruding structure; and
securing a liquid-tight structure to the hollow coiled shaft to define a fluid-delivery lumen within the internal space.

19. The method of claim 18, wherein securing the liquid-tight structure includes inserting a resin tube into the internal space and bonding a proximal end of the resin tube to the hollow coiled shaft.

20. The method of claim 16, wherein securing the liquid-tight structure includes coating the hollow coiled shaft with a resin material such that gaps between adjacent turns of the first wire are sealed.
